(19) United States
(12) Plant Patent Application Publication
(10) Pub. No.: US 2012/0291166 P1
(43) Pub. Date: Nov. 15, 2012

(54) Gerbera plant named 'UFGE 7015'

(50) ;

(76) Inventors: Zhanao Deng, Riverview, FL (US); Brent K. Harbaugh, (US)

(21) Appl. No.: 13/068,352

(22) Filed: May 9, 2011

(51) Int. Cl. A01H 5/00

(52) U.S. Cl. PLT/357

(57) ABSTRACT

A new Gerbera plant particularly distinguished by having a novel combination of medium lengths of peduncles, double bright white flower color, an overall flower diameter of approximately 10.5 cm, a high level of resistance to powdery mildew, and demonstrated potential to produce attractive plants in large (≧20-cm in diameter) containers, is disclosed.

### GENUS AND SPECIES

Gerbera hybrida

### VARIETY DENOMINATION

‘UFGE 7015’

### BACKGROUND OF THE NEW PLANT

The invention relates to a new and distinct variety of Gerbera hybrida plant named ‘UFGE 7015’. ‘UFGE 7015’ originated from a cross made at Wimauma, Fla. in 2005 between the female parent, ‘Sunburst Snow White’ (unpatented), and the male parent, ‘UFGE 4033’ (unpatented), an unreleased breeding line selected at Wimauma, Fla. in 2004 from a population of progeny of the cross ‘UFGE 31-19’ (unpatented) and ‘UFGE 35-4’ (unpatented). ‘UFGE 7015’ was selected by the inventors from the progeny of the stated parentage in summer 2007 at Wimauma, Fla. The first asexual reproduction of ‘UFGE 7015’ was accomplished when crown division was done in fall 2007 at Wimauma, Fla. Plants of ‘UFGE 7015’ have been asexually propagated by crown division and/or tissue culture for more than three generations. Asexually propagated plants of ‘UFGE 7015’ have remained true to the original selected plant, and all characteristics of the plant have been transmitted and retained through three successive asexual vegetative generations.

Plant Breeder's Rights for this cultivar have not been applied for. ‘UFGE 7015’ has not been made publicly available more than one year prior to the filing of this application.

### SUMMARY OF THE INVENTION

The new and distinct variety of Gerbera plant is characterized by a novel combination of medium lengths of peduncles, double bright white flowers, an overall flower diameter of approximately 10.5 cm, a high level of resistance to powdery mildew, and demonstrated potential to produce attractive plants in large (≧20-cm in diameter) containers. ‘UFGE 7015’ has not been observed under all possible environmental conditions. Its phenotype may vary significantly with variations in environment such as light intensity, temperature, and day length. The following are the most outstanding and distinguishing characteristics of this new cultivar when grown at Wimauma, Fla. under normal horticultural practices in greenhouse conditions which closely approximate those generally used in commercial practice.

1. Peduncle length of approximately 39 cm;

2. Double flower type;

3. White (RHS 155D) ray florets with a pink undertone (RHS 36D);

4. Yellow-green (RHS 151D) color discs before opening of disc florets;

5. White (RHS 155D) perianth lobe of disc florets;

6. Flower head diameter of approximately 10.5 cm; and

7. High resistance to powdery mildew.

### DESCRIPTION OF THE PHOTOGRAPHS

This new Gerbera plant is illustrated by the accompanying photographs which show the plant's form, flower head, and foliage. The colors shown are as true as can be reasonably obtained by conventional photographic procedures. The photographs are of a plant approximately 4 months old which was produced from one tissue culture liner and is potted in a 2.7-L container. Colors in the photograph may differ slightly from the color values cited in the detailed botanical description, which accurately describe the colors of the new Gerbera.

FIG. 1 shows the overall plant habit including flowers and foliage and is taken from a side perspective view.

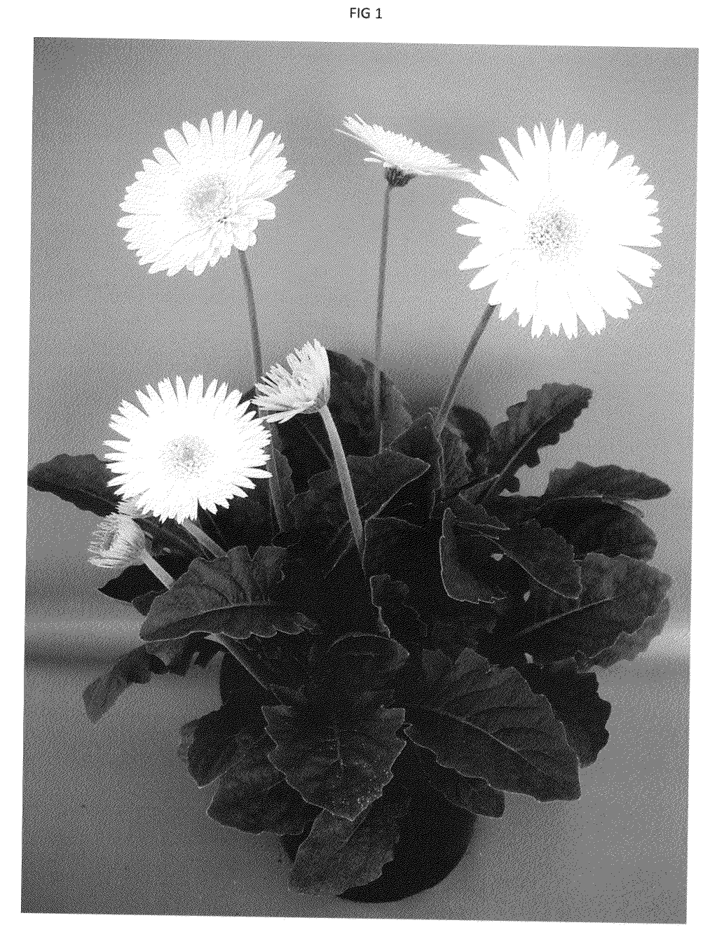

FIG. 2 shows a close-up of the flower head.

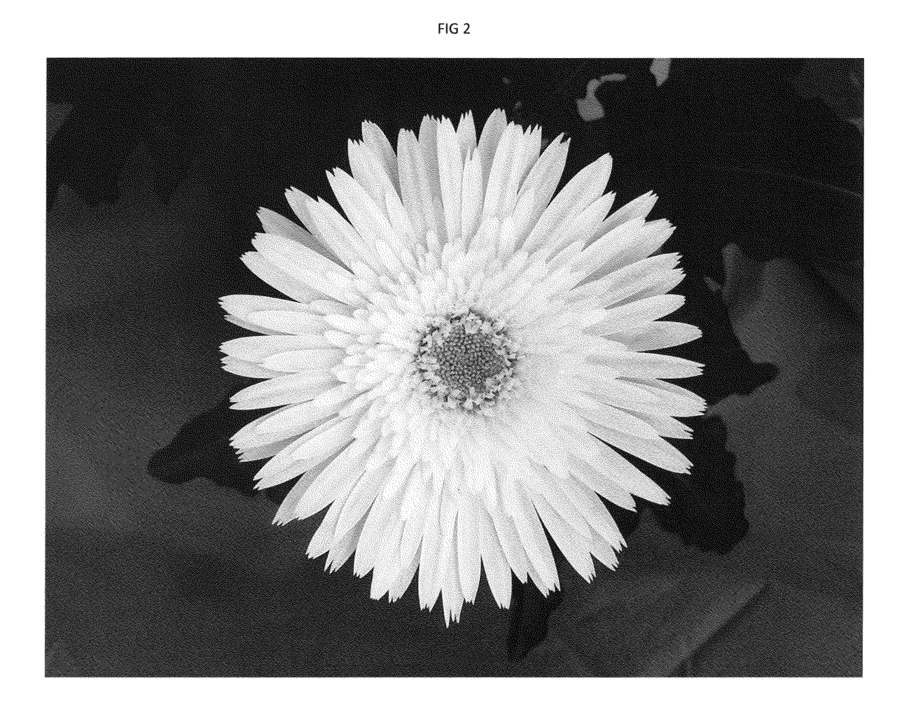

FIG. 3 shows a close-up of the typical leaf.

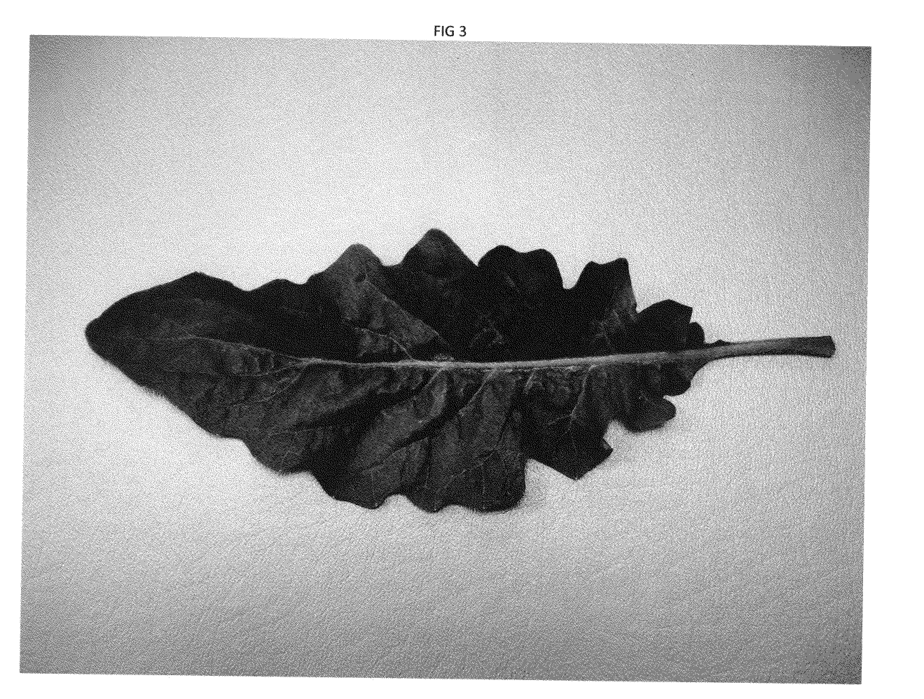

### DESCRIPTION OF THE NEW CULTIVAR

The following detailed description sets forth the distinctive characteristics of ‘UFGE 7015’. The present botanical description is that of the variety at approximately 4 months old on Nov. 23, 2009 at 11:30 a.m. in a room under north light at Wimauma, Fla. The colors (except those in common terms) are described from R.H.S. Colour Chart published by The Royal Horticultural Society in London (1986 ed.), in association with the Flower Council of Holland.

### DETAILED BOTANICAL DESCRIPTION

Classification:
   Botanical.—Gerbera hybrida cv. ‘UFGE 7015’.
   Common name.—Gerbera.
Parentage:
   Female parent.—‘Sunburst Snow White’ (unpatented).
   Male parent.—‘UFGE 4033’ (unpatented).
Inflorescence:
   Flower head.—Type: Double Diameter: Medium, approx. 105 mm Color (general tonality from a distance of 3 meters): White (RHS 155D) with a pink undertone (RHS 36D) Shape: Incurving funnel-shaped.
   Involucre.—Height from point of attachment of involucre to top of flower head: Short, approx. 19 mm Diameter: Small to medium, approx. 35 mm Number of bracts: Medium, approx. 87 Color: Green (RHS 138A) Longitudinal axis of inner rows: Straight Anthocyanin: Absent Pubescence: Very dense.
   Inner ray florets.—Number: Medium, approx. 58 Shape: Narrow elliptic Longitudinal axis of outer row: Straight Longitudinal axis of inner rows: Straight Longitudinal axis of ray female floret: Moderately incurving.
   Outer ray floret.—Cross section: Concave Length: Medium, approx. 39 mm Width: Medium, approx. 6-7 mm Shape of apex: Pointed Depth of incision: Medium-deep Color (topside): White (RHS 155D) Color (bottom side): White (RHS 155D) Color distribution on inner side: Different, red (RHS 36D) in middle of florets Edge of different color: Absent Striation: Present.
   Disc florets.—Disc diameter: Medium, approx. 26 mm.
   Reproductive organs.—Style (main color of distal part): White (RHS 155B) Stigma (main color): White (RHS 155D) Anthers (main color): Yellow (RHS 9A) Color of top relative to other parts: Lighter Longitudinal stripes: Absent.
   Pappus.—Main color: Yellow-white (RHS 158D) Color of top relative to other parts: Same Level of top relative to closed disc florets: Above, 1-2 mm in center.
   Peduncle.—Length: Medium, approx. 38 cm Tendency to fasciation: Absent Thickness: Medium, approx. 5 mm Strength: Medium Pubescence: Medium Color: Yellow-green (RHS 144B) Anthocyanin coloration: At base: Very weak At top: Absent Involucral bracts: Absent.
Plant:
   General appearance.—Height: Approx. 39 cm, excluding any flowers Width: Approx. 45 cm.
   Foliage.—Leaf blade: Length: Long, approx. 23 cm Width: Medium, approx. 11 cm Thickness: Medium Blistering: Weak Pubescence (On upper side, midrib excluded): Medium Depth of incisions in leaf: Basal part: Deep Central part: Medium Distal part: Shallow Color: Upper side: Green (RHS 137B) Bottom side: Yellow-green (RHS 146B) Glossiness on upper side: Strong Shape of apex: Moderately acute.
   Petiole.—Petiole length: Medium, approx. 7 cm Color of petiole: Yellow-green (RHS 144B) Petiole anthocyanin coloration: Strong.
Resistance to diseases: ‘UFGE 7015’ has been observed in multiple experiments in which it was compared with two common commercial cultivars, ‘Pensacola’ gerbera (unpatented) and ‘Bimini’ gerbera (unpatented), for the incidence and severity of powdery mildew, Podosphaera (syn. Sphaerotheca) fusca (Fr.) S. Blumer. ‘UFGE 7015’ has consistently been found to have a high level of resistance to powdery mildew. In one experiment (Experiment 1), conducted in winter 2008 and spring 2009 at Wimauma, Fla., tissue culture liners of ‘UFGE 7015’, ‘Bimini’, and ‘Pensacola’ were transplanted on into 2.7-L containers filled with commercial potting mix amended with controlled release fertilizer at the rate of 5.28 kg·m⁻³ and trace element fertilizer at the rate of 1.05 kg·m⁻³. Plants were grown under a plastic tunnel within a screen house with approximately 45% light exclusion. Fungicides were not applied throughout the experiments to subject the plants to natural powdery mildew disease pressures. In Experiment 1, the severity of powdery mildew on gerbera leaves was assessed at week 8 after transplanting (Feb. 4, 2009), week 10 after transplanting (Feb. 18, 2009), week 12 after transplanting (Mar. 4, 2009), and week 14 after transplanting (Mar. 18, 2009). A randomized complete block design was used with five replications. The experimental unit was a single containerized plant. ‘UFGE 7015’ showed a high level of resistance to powdery mildew in Experiment 1 (Table 1). ‘UFGE 7015’ remained to be more productive than ‘Bimini’ and ‘Pensacola’ when natural powdery mildew disease pressure existed (Table 1). No other disease resistance characterizations have been made.

TABLE 1
Powdery mildew severity ratings and flower counts of ‘UFGE 7015’
and two commercial gerbera cultivars, ‘Bimini’ and ‘Pensacola’,
grown under natural powdery mildew pressure in Experiment 1
(Winter 2008 through Spring 2009) in Wimauma, FL.

| | PM Ratings^{Z} | | | | Flower |
|---|---|---|---|---|---|
| Cultivars | Week 8 | Week 10 | Week 12 | Week 14 | counts^{Y} |
| | Experiment 1 (Winter 2008 through Spring 2009) | | | | |
| UFGE 7015 | 1.0 | 1.8 | 2.4 | 3.6 | 7.6 |
| Bimini | 1.2 | 1.8 | 5.4 | 8.0 | 3.2 |
| Pensacola | 1.0 | 2.0 | 4.8 | 8.6 | 3.0 |

^{Z}Powdery mildew severity was rated on a scale of 1 to 10 as described by Hausbeck et al. (2002): 1 = no disease, 2 = trace to 10%, 3 = 10% to 20%, 4 = 20 to 30%, 5 = 30% to 40%, 6 = 40% to 50%, 7 = 50% to 60%, 8 = 60% to 70%, 9 = 70% to 80%, and 10 = 80% to 100% of leaf surface covered with powdery mildew.
^{Y}Total number of flowers produced per plant over nine weeks from Jan. 20, 2009 to Mar. 17, 2009 in Experiment 1 in a screen house in Wimauma, FL

Comparision with parental and known cultivars: ‘UFGE 7015’ differs from the female parent ‘Sunburst Snow White’ (unpatented) by having tall (approximately 40 cm) plants, as opposed to short (approximately 20 cm) plants under growing conditions in Wimauma, Fla. ‘UFGE 7015’ differs from the male parent ‘UFGE 4033’ (unpatented) by having full double flowers, as opposed to single flowers under growing conditions in Wimauma, Fla. Comparisons were made with the commercial gerbera variety ‘Bimini’ (unpatented) and the commercial gerbera variety ‘Pensacola’ (unpatented). One experiment was conducted in a greenhouse at Wimauma, Fla. under standard greenhouse management practices to evaluate the plant performance (days to flower, flower quality, flower count, and plant quality) of ‘UFGE 7015’ for container plant production in comparison to ‘Bimini’ and ‘Pensacola’. In this plant performance experiment (Experiment 2), tissue culture liners were transplanted on Dec. 9, 2008 into 2.7-L containers filled with commercial potting mix amended with controlled release fertilizer at the rate of 5.28 kg·m⁻³ and trace element fertilizer at the rate of 1.05 kg·m⁻³. Potted plants were grown on metal benches in a glass house and spaced 46 cm×46 cm apart. Temperatures inside the greenhouse ranged from 18 ° C. to 33° C. Two hours of additional photoperiodic lighting were provided between Dec. 9, 2008 and Mar. 17, 2009. Fungicides and insecticides were applied as needed to control powdery mildew and western flower thrips. Flower quality was rated on a 1 to 5 scale: 1=very poor, 3=fair, some blemishes, but acceptable, and 5=excellent, bright, uniform, and no blemishes. Flower counts were recorded weekly from Jan. 20, 2009 through Mar. 17, 2009. Plant quality was rated three times, on Jan. 20, 2009, Feb. 3, 2009, and Feb. 17, 2009, using a 1 to 5 scale: 132 few leaves or long petioles, container surface visible, very poor and unacceptable as flowering pot plants, 3=fair and marketable, and 5=excellent, full, symmetrical, attractive plants. The experiment design for Experiment 2 was a randomized complete block design with five replications. The experimental unit was a single containerized plant. ‘UFGE 7015’ came into flowering 11.8 to 16.6 days earlier than ‘Bimini’ and ‘Pensacola’ in Experiment 2 (Table 2). The flower quality rating of ‘UFGE 7015’ was 4.5, similar to that of ‘Bimini’ (5.0), and higher than that of ‘Pensacola’ (4.1) (Table 2). ‘UFGE 7015’ produced more flowers (320% more than ‘Bimini’ and 686% more than ‘Pensacola’) (Table 2). The plant quality rating of ‘UFGE 7015’ was 4.8, higher than that of the controls (Table 2).

TABLE 2
Plant performance of ‘UFGE 7015’ and two commercial cultivars,
‘Bimini’ and ‘Pensacola’, grown in Experiment 2
(Winter 2008 through Spring 2009) in Wimauma, Florida.

| | Days to 1st open | Flower | Flower counts^{Z} | Plant |
|---|---|---|---|---|
| Cultivars | flowers (no.) | quality^{Z} | (no.) | quality^{Y} |
| | Experiment 2 (Dec. 9, 2008 to Mar. 17, 2009) | | | |
| UFGE 7015 | 52.2 | 4.5 | 9.6 | 4.8 |
| Bimini | 64.0 | 5.0 | 3.0 | 3.1 |
| Pensacola | 68.8 | 4.1 | 1.4 | 3.3 |

^{Z}Flower count data were collected over nine weeks from Jan. 20, 2009 to Mar. 17, 2009. Each value represents the mean of five replicates.
^{Y}The plant quality rating value was the mean of five replicates over three times (Jan. 20, Feb. 3, and Feb. 17, 2009).

Literature cited: Hausbeck, M. K., W. R. Quackenbush, and S. D. Linderman. 2002. Evaluation of cultivars of African daisy for resistance to powdery mildew, 2002. B&C Tests 18:00004.

I claim:

1. A new and distinct cultivar of Gerbera plant named ‘UFGE 7015’, as illustrated and described herein.
